(19) United States
(12) Patent Application Publication
(10) Pub. No.: US 2002/0127224 A1
(43) Pub. Date: Sep. 12, 2002

(54) Use of photoluminescent nanoparticles for photodynamic therapy

(76) Inventor: James Chen, Bellevue, WA (US)

Correspondence Address: Stephanie Seidman, Heller Ehrman White & McAuliffe LLP, 7th Floor, 4350 La Jolla Village Drive, San Diego, CA, 92122, US

(21) Appl. No.: 10/091,144

(22) Filed: Mar. 4, 2002

Related U.S. Application Data
(60) Provisional application No. 60/272,877, filed on Mar. 2, 2001.

(51) Int. Cl. A61K 39/395; A61K 9/50

(52) U.S. Cl. 424/130.1; 424/499

(57) ABSTRACT

Disclosed are compositions and methods that can be used to effect a photodynamic therapy (PDT) such as cancer treatment or gene transcription. Compositions include light-emitting nanoparticles that absorb light of one wavelength emitted by a light source and emit light of another wavelength that activates a PDT drug. Light-emitting nanoparticles include quantum dots, nanocrystals, and quantum rods as well as mixtures of these nanoparticles. The nanoparticles may be delivered to a patient in a liquid carrier or as part of a solid carrier such as a biocompatible polymeric film, a polymeric sheath, or other carrier suitable for introduction at the site to be treated. In one embodiment of the invention, light-emitting nanoparticles are localized at the treatment site by either joining them to the PDT drug covalently or non-covalently through linkage groups such as biotin/avidin, or the nanoparticles are localized at the treatment site by attaching the nanoparticles to a linkage group that has affinity for e.g. cells or proteins produced at the site to the treated. A sufficient number of light-emitting nanoparticles are delivered to the treatment site to activate the PDT drug and effect treatment.

### RELATED APPLICATIONS

Benefit of priority under 35 U.S.C. 119(e) is claimed to U.S. provisional application Serial No. 60/272,877, filed Mar. 2, 2001, to Chen, entitled “NOVEL USES OF PHOTOLUMINESCENT NANOPARTICLES FOR PHOTODYNAMIC THERAPY;” and to U.S. application Ser. No. 09/798,277, filed Mar. 2, 2001, and converted to a provisional application on Feb. 26, 2002, serial number unavailable, to Chen, entitled “NOVEL USES OF PHOTOLUMINESCENT NANOPARTICLES FOR PHOTODYNAMIC THERAPY.” The disclosures of the above-referenced applications are incorporated herein by reference in their entirety.

### FIELD OF THE INVENTION

The invention relates generally to photodynamic therapy (PDT), and more specifically to illumination systems, compositions and methods of using light-emitting nanoparticles to activate photosensitive compounds at the target site for PDT.

### BACKGROUND

PDT is a form of energy-activated therapy for destroying abnormal or diseased tissue. The procedure for this treatment includes administration of a photosensitive compounds (a PDT drug) to a patent, followed by illumination with light having a wavelength or waveband corresponding to a characteristic absorption wavelength of the photosensitive compound. Upon illumination, the photosensitive compound absorbs photons from the light source and transfers this energy to surrounding oxygen molecules. This in turn induces formation of singlet oxygen and other highly-reactive free radical species, leading to a number of biological effects, including damages to proteins, nucleic acids, lipids, and other cellular components, and often ultimately results in cell death.

PDT drugs may be administered to a patent by ingestion or injection or by applying the compound to a specific treatment site on the patent's body. These compounds characteristically accumulate at higher concentrations in rapidly-growing tissue, such as malignant tumors, than in normal tissue. Typically, after administering a PDT drug intravenously and then waiting a period of time, the drug clears from normal tissues and is preferentially retained by rapidly-growing tissues. The drug remains inactive until exposed to light. Application of light of a suitable wavelength photoactivates the drug, resulting in generation of reactive species and damage to neighboring tissue. PDT has been used to treat various types of malignant tumors as well as non-cancerous condition such as macular degeneration and atherosclerosis.

Light sources utilized for PDT include monochromatic lasers linked to fiber optics and light emitting diode (LED) arrays. One disadvantage of such light sources is that they are not capable of broadband emission at multiple wavelengths or wavebands at which a drug can be activated. Often a PDT drug can be activated at more than one wavelength. To obtain light of multiple wavelengths, light from multiple lasers and/or from multiple LEDs must be coupled into a fiber optic. Lasers can be bulky, requiring in-office or in-hospital administration of light and requiring a significant amount of valuable space to house the multiple lasers. Further LEDs may not provide all wavelengths desired. The technology to provide LEDs producing blue, violet, and ultraviolet light is developing, but LEDs are not yet available to provide the full spectrum of specific wavelengths that can be useful to active PDT drugs. Further, light in a waveband from the blue to ultraviolet part of the spectrum does not penetrate tissue very deeply. Consequently, any light administered via laser or LED array in this portion of the spectrum only penetrates shallow portions of tissue at the site where light is introduced to the body.

### SUMMARY OF THE INVENTION

The invention provides a PDT therapy which is capable of utilizing light that deeply penetrates tissue, yet activates a wide range of PDT drugs, including those that activate at shorter wavelengths in which light will penetrate only shallow portions of tissue. The invention also provides a PDT therapy in which multiple PDT drugs requiring light of diverse wavelengths can be used without having to resort to multiple lasers or LED arrays. In addition, the invention provides a surgeon with the capability to select from a wide array of PDT drugs, including those whose use has been restricted or essentially prevented because of the short wavelength at which they absorb light.

The invention in one embodiment provides a photodynamic therapy in which light-emitting nanoparticles are administered to a patient in addition to a PDT drug in order to activate the drug. The light-emitting nanoparticles absorb light from the light source and re-emit light at a different wavelength, one which is suitable to activate the PDT drug in the vicinity of the light emitting nanoparticles. The PDT drug near the light-emitting nanoparticles is activated, thus treating the disease anyplace that the PDT drug and nanoparticles are located and that light from the light from the light source can reach.

The combination of light-emitting nanoparticles and PDT drug enables treatment of diseases using a broader array of PDT drugs and therapies than is presently available. For example, in another embodiment of the invention, a large tumor can be treated with a PDT drug which is activated by a wavelength of light that penetrates shallowly into tissue (e.g. less than 1 cm). The light-emitting nanoparticles are selected so that they absorb light having a wavelength that penetrates deeply into tissue and then re-emit light at a wavelength that activates the PDT drug. Providing such nanoparticles in conjunction with the PDT drug enables a significant portion of the depth of a large tumor to be treated, whereas with the previously-known PDT therapy only the very surface of the tumor might be amenable to treatment.

The light-emitting nanoparticles administered with a PDT drug may be activated by single photon absorption of activating light. Alternatively, the light-emitting nanoparticles may be activated by two photon absorption of the activating light. Nanoparticles that absorb two photons often emit light at a wavelength that is about equal to or longer than the wavelength of the activating light, whereas nanoparticles that absorb a single photon typically emit light at a wavelength that is about equal to or shorter than the wavelength of the activating light. PDT drug-activating light of a desired wavelength, wavelengths, or waveband can therefore be provided by selecting the size and composition of the quantum dots as well as the wavelength or wavelengths of laser light used to illuminate the quantum dots.

A light-emitting nanoparticle that emits a wavelength useful in PDT can be modified to have one or more functional moieties. A functional moiety can provide better solubility in polar or aqueous materials or in organic materials. A functional moiety may also be a delivery moiety such as a particular protein, oligo- or polynucleotide sequence, antibody, or organism that place the light-emitting nanoparticle at or near the PDT treatment site.

Light-emitting nanoparticles can be used in any photodynamic therapy, not just tumor treatment. Consequently, light-emitting nanoparticles can be used in PDT to activate gene transcription, treat psoriasis, and treat macular degeneration among other diseases.

A light-emitting nanoparticle may be stimulated to emit light using light of a different wavelength that has been focused upon it by a total internal reflection lens. A total internal reflection lens concentrates light from a light source upon a desired area such as a tumor at which e.g. quantum dots or quantum rods are present, thus assuring that more of the light provided by the light source stimulates the nanoparticles to fluoresce at the treatment site. Thus, any of the embodiments of the invention as described herein, including illumination systems and methods, may utilize or incorporate a total internal reflection lens as one of the elements focusing light from the light source.

Further, light from light-emitting nanoparticles may be focused upon the treatment site using a total internal reflection lens. Thus, the light emitted by such light-emitting nanoparticles as quantum dots or quantum rods is concentrated upon a desired area such as a tumor, thus assuring that more of the light emitted by the nanoparticles activates one or more PDT drugs present at the treatment site. Thus, any of the embodiments of the invention as described here, including illumination systems and methods, may utilize or incorporate a total internal reflection lens as one of the elements focusing light from the light-emitting nanoparticles to the site to be treated, either with an additional total internal reflection lens focusing light from the light source upon these nanoparticles or without the additional total internal reflection lens.

The invention further provides an illumination system comprising a light source and light-emitting nanoparticles as well as an illumination system comprising a light source and a total internal reflection lens that is configured to be positioned with the patient.

Among other factors, the invention is based on the technical finding that a sufficient quantity of light-emitting nanoparticles in sufficient proximity to a PDT drug can activate the PDT drug, thereby generating a sufficient number of radicals such as singlet oxygen that a therapy such as tumor treatment or gene transcription is effected. The light-emitting nanoparticles are generally much closer to critical areas within the site to be treated than conventional light sources can be positioned. Consequently, the depth of the site at which e.g. necrosis or transcription occurs beneath the skin is markedly greater than is possible with conventional external PDT therapies. It is also possible to treat a greater thickness or depth of tumor or other tissue because light-emitting nanoparticles can be distributed within the tissue to illuminate large portions of it and because activating light which penetrates tissue deeply but is otherwise poor at activating a PDT drug can be used to stimulate the nanoparticles to fluoresce. Light-emitting nanoparticles also allow a wider range of PDT drugs and therapies to be used, since the wavelength at which a PDT drug functions does not limit how the drug can be used. Further, light-emitting nanoparticles can activate a given PDT drug at multiple wavelengths simultaneously, imparting greater energy to the drug, and multiple drugs that activate at different wavelengths can be administered simultaneously when light-emitting nanoparticles are administered. Light-emitting nanoparticles having a delivery moiety attached or inserted at the treatment site also allow a PDT therapy to be focused or limited to the target site. Yet, tissue intermediate to e.g. a tumor and the surface of the skin is not affected by photodynamic therapy, even where the PDT drug is present in the intermediate tissue, where the wavelength of light absorbed by nanoparticles with delivery moieties does not activate the PDT drug. These technical findings and advantages and others are apparent from the discussion herein.

The invention thus provides a number of systems, components, compositions, means, and methods associated with photodynamic therapy and light-emitting nanoparticles as are more fully described below. This Summary section of the disclosure provides a summary of some salient points of the invention, but this section is not to be interpreted as limited the scope of the invention to only those features and embodiments discussed in this section. Instead, the invention involves all components, systems, and methods discussed in this and the following section that relate to compositions, systems, and methods involving light-emitting nanoparticles for PDT therapy in addition to those defined by the appended claims.

### BRIEF DESCRIPTION OF THE DRAWINGS

FIG. 1 illustrates a scheme for attachment of a protein molecule to a nanoparticle.

FIG. 2 is a view of a tumor blood vessel, showing an antibody-linked nanoparticle in the lumen of the blood vessel, bound to an antigen on the interior of the surface vessel.

FIG. 3 illustrates light emitting nanoparticles with a biotin delivery moiety and a protocol for associating multiple nanoparticles with an individual cancer cell.

FIG. 4 depicts PDT treatment of tumor tissue using an external and/or interstitial light source to illuminate nanoparticles localized at the treatment site.

FIG. 5A illustrates insertion of an optical fiber composed of a biocompatible matrix with incorporated nanoparticles. An external LED or laser source provides light to the fiber.

FIG. 5B shows the optical fiber after use. The external portion of the fiber is cut off flush with the skin and buried.

FIG. 6A illustrates insertion of a polymeric sheath containing incorporated nanoparticles, using a removable needle.

FIG. 6B illustrates the sheath after the needle has been removed and a fiber optic light source inserted in its place.

FIG. 7A illustrates an optic fiber having a total internal reflection (TIR) lens at its end, inserted through a polymeric sheath in which are embedded nanoparticles. The polymer is preferably transparent to the light from the light source as well as the light emitted by the nanoparticles.

FIG. 7B depicts an optical fiber inserted into a polymeric sheath having a TIR lens at its end. The TIR lens captures light emitted by nanoparticles that would otherwise illuminate healthy tissue and directs it to diseases tissue.

FIGS. 8A and 8B depict an optic fiber equipped with a TIR lens and having nanoparticles embedded in the end of the fiber (and/or coated onto the TIR lens along the optic path of the lens).

### DETAILED DESCRIPTION OF PREFERRED EMBODIMENTS

The invention provides a number of compositions, methods, and components in which light-emitting nanoparticles are used in photodynamic therapy. The following discussion provides further details on various aspects of the invention as well as discussion of certain preferred embodiments of the invention. The invention is, of course, not limited to the preferred embodiments but it is instead to be accorded a scope consistent with the disclosed general principles as well as the various embodiments discussed herein. All patents and other references referred to herein are incorporated by reference in their entirety.

The invention in one embodiment involves administering therapeutically effective amounts of both photosensitive compound as used in photodynamic therapy and light-emitting nanoparticles to a patient, either separately or simultaneously. A light source producing light of a first wavelength illuminates the patient, externally or internally (or both). The light-emitting nanoparticles absorb this light and then emit light of a second wavelength. The photosensitive compound absorbs light of the second wavelength and converts the energy imparted to the compound (a drug) into chemical energy by producing a reactive species such as singlet oxygen. The reactive species then either directly or indirectly causes a desired event (such as cell necrosis or apoptosis or gene transcription).

Prior to discussing therapies in which light-emitting nanoparticles are utilized, light-emitting nanoparticles and various forms they may take are described.

1. Light-Emitting Nanoparticles

There are a number of light-emitting nanoparticles that my be used, alone or in combination with one another, in photodynamic therapy. These include quantum dots, nanocrystals, and quantum rods.

1a) Quantum Dots

Quantum dots are small molecular clusters having up to about a few hundred atoms. Quantum dots are therefore larger than individual atoms, but quantum dots generally behave in accord with the principles of quantum mechanics that govern the behavior of individual atoms. Because of this behavior, quantum dots are sometimes also called “artificial atoms.” Quantum dots have a size in the regime of about 1 nm to about 20 nm and are typically only a few nanometers in size.

A quantum dot is typically composed of a semiconductor material or materials, metal(s), or metal oxides exhibiting a certain bandgap energy. Although it is preferred that biocompatible light-emitting nanoparticles such as TiO₂ are used in the practice of the invention, nanoparticles that are not generally considered to be biocompatible may also be used. A variety of materials may be utilized for construction of nanoparticles, including but not limited to TiO₂, Al₂O₃, AgBr, CdSe, CdS, CdS_{X}Se_{1-X}, CuC1, CdTe_{X}S_{1-X}, ZnTe, ZnSe, ZnS, GaN, InGaN, InP, CdS/HgS/CdS, and InAs/GaAs. Group II-VI, Groups III-V, and Groups I-VII semiconductors as well as Group IV metals and alloys from quantum dots and other nanoparticles as described below when formed sufficiently small. A quantum dot may also be surrounded by a material or materials having wider bandgap energies (for example, ZnS-capped CdS), and especially may be surrounded by those materials that improve biocompatibility of the nanoparticles.

Quantum dots photoluminesce when stimulated by light having a wavelength in which the energy of a photon is at least equal to the bandgap energy of the light-emitting material forming the quantum dot. Consequently, quantum dots absorb light of a first wavelength and emit light at a second wavelength that is shorter than the first wavelength. The pump light supplied by e.g. a laser or light-emitting diode (“LED”) array or other light source in which photons have an energy at least equal to the band-gap energy of the quantum dot is therefore absorbed by the quantum dot. The quantum dot re-emits energy in the form of light at a different wavelength and in a multidirectional fashion.

Because a quantum dot is so small and is thus governed by the rules of quantum mechanics, the size of the dot dictates the wavelength, and hence the color, of its fluorescence. The larger the diameter or width of a given quantum dot, the longer the wavelength of light that the quantum dot emits. For example, the band gap of a CdSe quantum dot can be tuned from deep red (1.7 eV) to green (2.4 eV) by reducing the diameter of the quantum dot from 200 Å to 20 Å.

There are a number of methods of making quantum dots. The synthesis of small semiconductor clusters in trioctylphosphine oxide (TOPO) at 300° C. has been shown to yield highly fluorescent (quantum yields >50%) small particles of a number of semiconductor materials, such as CdSe, InP and InAs. Growth conditions such as the length of time of crystallization, concentration of monomer, and temperature establish the size of the quantum dot and therefore the color of the light emitted from the quantum dot. Such methods are discussed in the article by M. Green and P. O'Brien, “Recent advances in the preparation of semiconductors as isolated nanometric particles: new routes to quantum dots,” Chem. Commun., 1999, 2235-41 (“Green et al. article”). See also U.S. Pat. No. 5,909,670, 5,943,354, and 5,882,779.

1b) Nanocrystals

A nanocrystal is a molecular cluster of quantum dots that is large enough to have an identifiable interior, structurally identical to the corresponding bulk solid but having a substantial fraction of the total number of atoms on the surface. A nanocrystal has a size from about 20 nm to a size in which quantum effects no longer predominate (that is, where the wavelength of light emitted is not a function of the diameter or size of the particle). Nanocrystals are generally spherical or are irregularly shaped. Nanocrystals may also be formed as disclosed in the Green et al. article as well as the patents cited above for quantum dots and other methods known in the art.

1c) Quantum Rods

A quantum rod is a structure formed of quantum dots in which the diameter or width of the rod exhibits quantum effects (that is, the wavelength of light emitted is a function of the diameter or width), but the rod has a length that is greater than that which contributes to the quantum effect (i.e., where the length has no effect on the wavelength of light emitted). Quantum rods emit much brighter light than individual quantum dots or nanocrystals formed of the same material.

Quantum rods can be made by controlling the growth kinetics of nanocrystals. One method of making quantum rods involves using both a very high concentration of monomer during crystal growth and a surfactant that allows for a controlled growth rate of crystalline structure. The article by X. Peng, L. Manna, W. Yang, J. Wickham, E. Scher, A. Kavanich & A. Alivisatos, “Shape control of CdSe nanocrystals,” Nature, V. 404, Mar. 2, 2000, pp. 59 et seq. (“Peng et al. Article”) discloses one such method.

1d) Superlattice

A three-dimensional array of quantum dots may be formed and used in accordance with the principles of the invention. For example, colloidal CdSe nanocrystals that are passivated with organic surfactants and size-selected to a very high degree so that they are of essentially uniform size will spontaneously precipitate from solution to form the array. The array can have bands of electronic states, as described in the article by A. Alivisatos, “Semiconductor Clusters, Nanocrystals, and Quantum Dots,” Science, V 271, Feb. 16, 1996, 933-37 and the article by C. Murray, C. Kagan, & M. Bawendi, Science, V.270, 1995, pp. 1335 et seq.

1e) Mixtures

Mixtures of quantum dots, nanocrystals, and/or quantum rods can be formed to match specific applications. For example, quantum rods of various sizes and compositions may be mixed to provide light of different wavelengths. The quantum rods of the mixture are selected based on their capacity to activate one or more selected photoactive compounds. For example, a number of PDT drugs absorb light and activate at two or more wavelengths. Consequently, quantum rods that emit those wavelengths can be mixed together and used to activate a PDT drug. These quantum rods can be formed of the same material (e.g. CdSe) but have different particle sizes that are selected to provide the appropriate wavelengths, or these quantum rods may be formed of different materials and have individual particle sizes that produce the wavelengths that activate the drug or drugs.

If desired, quantum rods of different diameter and optionally different lengths formed of the same material may be mixed together to provide light within a desired waveband or rage of wavelengths, or quantum rods of approximately the same size but made of different materials can be mixed to provide the same waveband of light. A mixture of quantum rods that provides one or more wavebands of light is also useful to accommodate small variations in activation wavelength for a particular drug, where the photoactivation wavelength varies slightly from one molecule of the drug to the next molecule of that drug.

Quantum dots may be mixed with nanocrystals and/or quantum rods to provide a composition that emits light of multiple selected wavelengths, with the intensity of light emitted at one wavelength differing from the intensity of light emitted at a second wavelength. In this instance, nanocrystals would usually produce light of greater intensity than light produced by quantum dots, and quantum rods would usually produce light of greater intensity than light produced by nanocrystals formed of the same light-emitting material.

PDT drugs are often subject to “bleaching,” a phenomenon in which the drugs lose their effectiveness as a function of the light intensity used to activate the drug and the number of activation “cycles” that the drug experiences. Different drugs have different bleaching rates, and thus if a plurality of PDT drugs are used in combination in photodynamic therapy, different intensities of light at the various wavelengths may help to present premature bleaching. Further, even though a particular drug activates at multiple wavelengths, the amount of light absorbed at one wavelength usually differs from the amount of light that is absorbed at another wavelength for this drug. Consequently, it can be useful to provide a mixture of nanoparticles that provide a waveband of light in which the intensities of certain wavelengths within the waveband differ based on the drug or drugs to be employed in the photodynamic therapy.

The amount of light generated at a given wavelength can be selected by adjusting the length of a quantum rod. A quantum rod equal in diameter and material of construction to another quantum rod but made longer than the other quantum rod will produce a greater illuminance (flux per illuminated unit area) and fluence (time integrated flux of photons per illuminated area) of light. Therefore, a mixture of quantum rods of various lengths and diameters can be used to provide a light illuminance or fluence that varies by wavelength within the waveband generated by the mixture. Further, quantum rods can be mixed with quantum dots and/or nanocrystals to vary the illuminance or fluence of light as a function of the wavelength emitted.

A mixture of quantum dots, nanocrystals, and/or quantum rods may be selected to provide certain desired wavelengths of light, or the mixture may be selected to provide a substantially continuous spectrum of light between two wavelengths. Further, the mixture can be selected to provide plural wavebands of emitted light. For example, a first light-emitting material is used to form nanoparticles (quantum dots, nanocrystals, and/or quantum rods), and the size of the particles is selected to provide light of a continuous or discrete spectrum of approximately 20 nm around a second wavelength at which either the same drug or a different PDT drug activates. The two sets of nanoparticles are mixed to form the mixture. This mixture of nanoparticles thus emits light having wavelengths within two wavebands that are centered about the wavelengths at which a PDT drug or drugs activate.

The light-emitting nanoparticles of the mixture can also be selected so that they absorb light of a single wavelength or narrow waveband, as is produced by a laser, or the nanoparticles can be selected so that they absorb different wavelengths of light as supplied by a light source that produces a wider range of wavelengths, such as that light supplied by an LED array. Thus, a mixture of particles can absorb light emitted within a narrow waveband and emit light within a narrow waveband, a broad waveband, or within multiple wavebands by selecting the materials forming the individual nanoparticles, their sizes, and/or their shapes as described above.

The various quantum dots, nanocrystals, and/or quantum rods may be mixed dry or in a liquid or gaseous carrier using methods known in the art for mixing small particles. Such carriers include blood, a blood product such as plasma, an oil, an ointment, a cream, or a gel.

2. Light-Emitting Nanoparticle having a Group to Solubilize the Nanoparticle

The light-emitting nanoparticles discussed above may have hydrophilic groups attached to them in order to render the nanoparticles soluble in polar solvents. One such procedure is disclosed in Chan and Nie, Science 281, p. 2016 (1998) (“Chan et al. Article”). This procedure involves use of mercaptoacetic acid to solubilize ZnS-capped quantum dots. When the reaction is performed in chloroform, the mercapto groups bind to the Zn atoms, and the free polar carboxylic acid groups render the quantum dots water-soluble.

Instead of using mercaptoacetic acid in a reaction with light-emitting nanoparticles, a mercapto-organic material can instead be used to improve the solubility of light-emitting nanoparticles in organic liquids. Likewise, an amphipathic material, which improves nanoparticle solubility in either a polar or non-polar solvent, can be attached to a light-emitting nanoparticle through a mercapto group or other reactive group.

3. Light-Emitting Nanoparticle having a Delivery Moiety

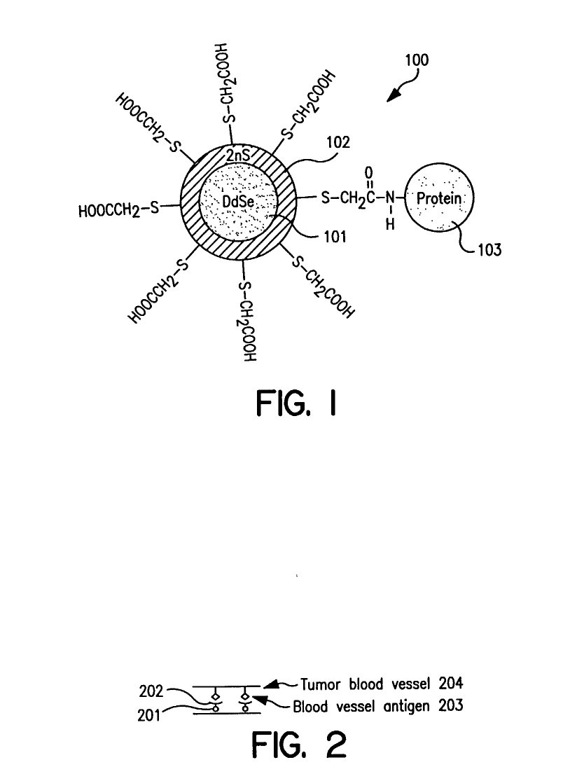

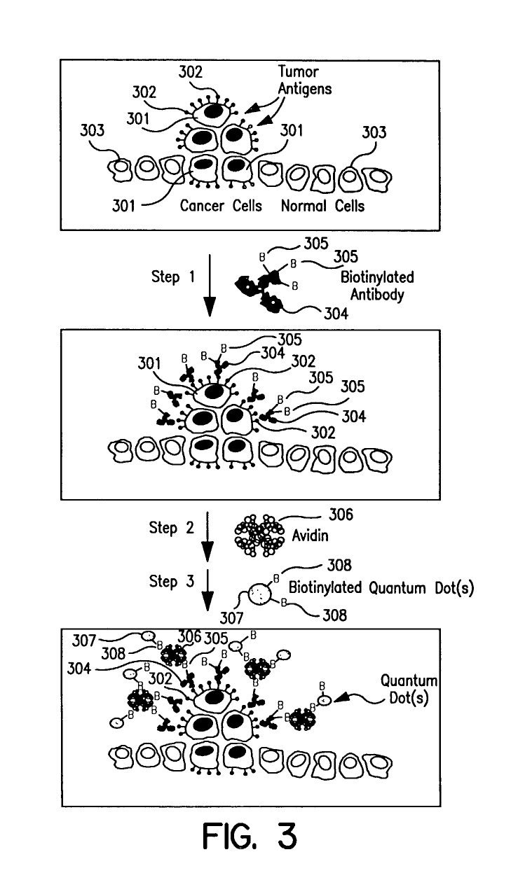

A light-emitting nanoparticle may be attached to a delivery moiety, which delivers the nanoparticle to a specific target site within the patient. A delivery moiety may be a protein, an oligo- or polynucleic acid, an antibody, a drug, an enzyme, an ion of high affinity for the target site, or other moiety that binds or complexes with the target site or a component found at the target site. FIG. 1 illustrates this type of nanoparticle 100, in which a DdSe quantum dot 101 is coated with ZnS 102, which has a wider or larger bandgap than CdSe has, and which is attached to protein 103 through a mercapto-acetic acid group that has reacted with a free amine on the protein. Especially preferred delivery moieties are antibodies that are specific to a site to be treated with PDT. For example, FIG. 2 illustrates a quantum dot 201 attached to an antibody 202 that binds to a blood vessel antigen 203 produced at a blood vessel 204 within a tumor. The antibody binds to the antigen, positioning the quantum dot or dots attached to the antibody at the site to be treated.

Examples of delivery moieties include endogenous inhibitors of angiogenesis such as angiostatin (Entremed, Rockville, Md.), Troponin-1 (Boston Life Sciences, Boston, Mass.), endostatin, TNP-470 (6-O-chioroacetylcarbonyl)-fumagillol (TOP Pharmaceuticals, Deerfield, Ill.), 2-methoxyestradiol, troponin-1, antithrombin, thrombospondin, metallospondins such as the proteins produced by the genes METH-1 and METH-2, tyrosine kinases such as Tie-1 and Tie-2, TNP-470 (a synthetic analog of the antibiotic fumagillin), squalamine (Magainin Pharmaceuticals, Plymouth Meeting, Pa.), indirect angiogenesis inhibitors such as IM-862 (a two amino-acid synthetic version of a natural peptide that upregulates interleukin-12 and inhibits vascular endothelial growth factor and basic fibroblast growth factor expression, Cytran, Kirkland, Wash.), interferon-α, interleukin-12 (Roche, Nutley, N.J.), and pentosan polysulfate (Georgetown University, Washington, DC), combretastatin A-4 (OXiGENE, Boston, Mass.) thalidomide (Celgene, Warren, N.J.), ZD-0101 (AstraZeneca, London, UK), therapeutics that target integrins such as αᵥβ₃ and αᵥβ₅ antagonists, antagonists that bind the angiogenic stimulator VEGF (such as those developed by Genentech, S. San Francisco, Calif.), other small molecules that block growth factor receptors such as Angiozyme (Ribozyme, Boulder, Colo.), PTK-787 and ZK-225846 (Novartis, Basil, Switzerland), and SU-5416, SU-101, and SU-6668 (Sugen, S. San Francisco, Calif.), proteolysis inhibitors such as Neovastat (Aeterna, Ste. Foy, Quebec), Metastat (Collagenex, Newtown, Pa.), Marimastat (British Biotech, Oxford, UK), CGS-27023A (Novartis, E. Hanover, N.J.), BMS-275291 (Bristol-Myers Squibb, Princeton, N.J.), Bay-12-9566 (Bayer, West Haven, Conn.), AG-3340 (Agouron, La Jolla, Calif.), αᵥβ₃ trichosanthin, molecules that block EC-ECM adhesion such as EMD-121974 (Merck KcgaA, Darmstadt, Germany) and Vitaxin (Ixsys, La Jolla, Calif.).

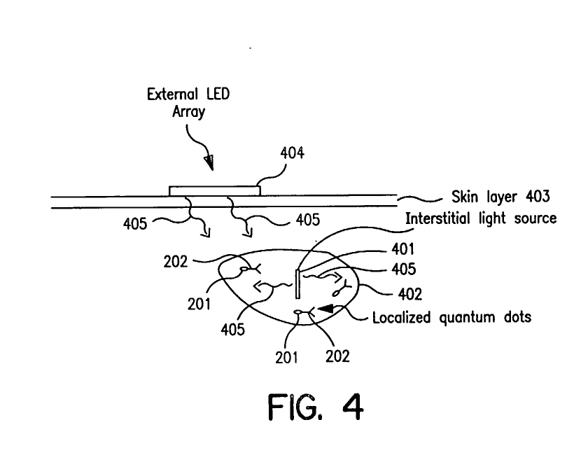

FIG. 3 illustrates how many light-emitting nanoparticles may be targeted to each cancer cell 301 without associating the nanoparticles with normal cells 303 by targeting the nanoparticles to antigens 302 on the cancer cells. An antibody 304 carrying multiple biotin molecules 305 is administered to the patient, and subsequently avidin or streptavidin 306 is administered to bind the biotin molecules carried by the antibody. Once the avidin or streptavidin molecule is bound to the biotinylated antibody, light-emitting nanoparticles 307 having one or more biotin molecule delivery groups 308 attached are administered. Each avidin or streptavidin molecule can bind multiple biotinylated light-emitting nanoparticles. Consequently, a plurality of light-emitting nanoparticles can be associated with each cancer cell.

Other biotinylated site-specific materials such as monoclonal antibodies, polyclonal antibodies, proteins, and oligo- or polynucleotides such as Fab fragments or proteins can be used in place of the antibody which binds to a tumor antigen in order to provide a wide range of target-specific components. Consequently, one member of an affinity binding set can be attached to a nanoparticle, and the other member of the affinity binding set may be a site-specific component. Affinity binding sets include: biotin-avidin; biotin-streptavidin; antibody-antigen; antibody fragment-antigen; chemokine-chemokine receptor; and growth factor-growth factor receptor.

The Chan et al. Article discusses a way of attaching quantum dots having free carboxylic acid groups to a molecule such as a protein. In this method, a carboxylic acid group on a quantum dot reacts with a free amine group to form an amide or peptide group. Nanocrystals and quantum rods may likewise be attached to a protein or other target-specific component using this method.

Another method of attaching quantum dots or other nanoparticles to a delivery moiety is disclosed in U.S. Pat. 5,990,479 (“'479 patent). The method of the '479 patent involves coating the nanoparticle with e.g. a thin coating (0.5 to 2 nm) of silica glass and linking the nanoparticle to a delivery moiety. The glass is linked to the nanoparticle through a linking agent such as substituted silane (e.g. 3-mercaptopropyltrimethoxysilane), and the delivery moiety is linked to the glass through a second linking agent such as an amine (e.g. 3-aminopropyltrimethoxysilane). Other methods of attaching a nanoparticle to a delivery moiety are discussed in Z. Chunyang et al., “Quantum DotLabelled Trichosanthin”, Analyst, 2000, 125, pp. 1029-1031 (quantum dot-labelled trichosanthin); L. Cattel et al., “The role of conjugation processes and linking agents in the preparation of molecular/particulate conjugates—a review,” S.T.P. Pharma Sciences 9 (4), pp. 307-319 (1999); WO 0029617; WO 0027365; and U.S. Pat. No. 5,055,446 and 5,985,353.

Coating light-emitting nanoparticles with silica or another material transparent to the wavelengths of light absorbed and emitted by the nanoparticles and that does not react to a significant extent with bodily fluids provides a way to improve biocompatibility of the nanoparticles. Consequently, nanoparticles that might otherwise not be considered suitable for use in a human can be coated to improve their biocompatibility.

4. Light-Emitting Nanoparticle Linked to PDT Drug

Light-emitting nanoparticles may be linked to a reactive group on a PDT drug by any of the methods described previously. Consequently, light-emitting nanoparticles may be covalently bound or may be complexed with the drug through a delivery moiety. The light-emitting nanoparticles are therefore positioned sufficiently closely to the PDT drug to illuminate it, even in instances where the tissue penetration depth of the light emitted by the nanoparticles is very small. The number of nanoparticles attached to the drug can also be selected to provide a sufficiently high concentration of nanoparticles around the PDT drug to assure the drug receives the intensity of light needed to activate the drug to provide the desired therapy.

Further, both the PDT drug and the light-emitting nanoparticles may be linked to an oligomeric or polymeric backbone. A polymer having free amine groups such as a melamine formaldehyde resin will react with both a PDT drug and nanoparticles that have been functionalized with carboxylic acid groups, and the PDT drug and nanoparticles can be spaced sufficiently closely to one another along the polymer that the nanoparticles assure that the PDT drug is illuminated.

5. Encapsulated Light-Emitting Nanoparticle

An alternate procedure for delivering nanoparticles to the treatment site involves encapsulating them in liposomes, microcapsules or nanocapsules, or other such drug carriers. Methods known to those in this field for incorporating small particles of a size similar to that of lightemitting nanoparticles can be utilized (see, e.g.; U.S. Pat. No. 5,686,113). Nanocapsules may be targeted to the treatment site by attachment of functional or delivery moieties on their outer surfaces as described above, such as antibodies or other delivery moieties. See also L. Cattel et al., “The role of conjugation processes and linking agents in the preparation of molecular/particulate conjugates—a review,” S.T.P. Pharma Sciences 9 (4), pp. 307-319 (1999).

6. Carrier for Light-Emitting Nanoparticles

a) Liquid, Emulsion, Gel, or Other Fluid Containing Nanoparticles

Any of the components discussed above (e.g. light-emitting nanoparticles, light-emitting nanoparticles having a solubilizing group, light-emitting nanoparticles having a delivery moiety, and encapsulated light-emitting nanoparticles) can be placed in a carrier that is suitable for delivery to the site to be treated with PDT. Consequently, these components may be formulated with a sterile or biocompatible carrier suitable for introduction into a human such as saline, caster oil, blood plasma or plasma-containing liquids, purified water, or other liquid carrier such as a bodily fluid that is present in the area to be treated. Topical carriers include ointments, creams, or gels that may be applied to the skin.

Other compositions containing the above-discussed components may be formulated to include drugs such as one or more PDT drugs, antibiotics, heparin, chemotherapy agents, therapeutic radionucleides, and other drugs whose efficacy is not substantially diminished by the presence of the light-emitting nanoparticles and which do not interfere substantially with light emission by the nanoparticles.

b. Immobilized Light-Emitting Nanoparticles

Immobilized light-emitting nanoparticles can be introduced to an area by applying them topically or by inserting or implanting the immobilized nanoparticles into the body through an incision or via injection or by another known method. Immobilized light-emitting nanoparticles are not free to move on or within the body when first introduced, as the other forms of nanoparticles described above are able to move when first introduced into or on the body. Immobilized in or on a matrix formed of a grid, fiber, fabric or other substrate that does not move when applied on or in the body. The matrix may be solid but transparent to the wavelengths at which the nanoparticles absorb and emit light, or the matrix may be porous so that nanoparticles are embedded within pores that transport light to and from the nanoparticles. The matrix also may be solid and translucent or opaque to the wavelengths at which the nanoparticles absorb and emit light where the nanoparticles are placed on the surface of the matrix. Shapes and forms for the matrix used to carry the immobilized nanoparticles include but are not limited to a fibrous shape, a rod-like or cylindrical shape, a grid, or a film.

The PDT drug may also form part of the matrix. The PDT drug may be absorbed into the matrix, or molecules of the PDT drug may be attached to the surface of the matrix so that the drug and nanoparticles are administered simultaneously.

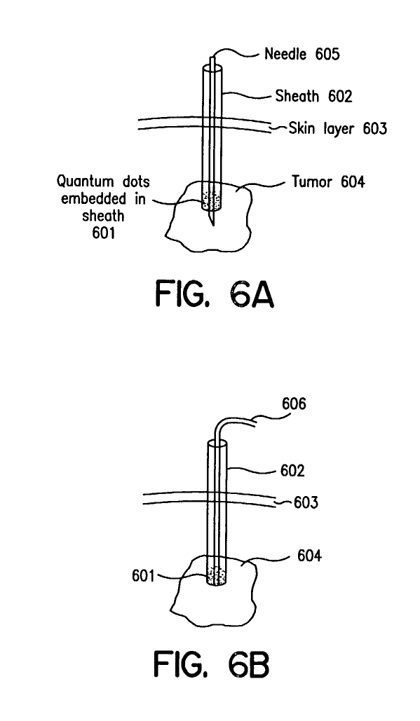

One example of the invention is shown in FIGS. 5A and 5B. An optical fiber 501 composed of a biocompatible matrix is shown. Quantum dots 502 coat the external shell of the fiber or are embedded in the matrix. The fiber is inserted beneath a patient's skin 503 in proximity to the PDT treatment site. An external light source, such as an LED, a laser, a laser diode, or other non-coherent light source such as a incandescent lamp or halogen light, pumps light into the fiber during treatment. After the treatment is complete, the externally exiting portion of the fiber is cut off flush with the skin 503 and buried, as illustrated in FIG. 5B. The fiber degrades over time and is reabsorbed. The optical fiber can be formed of a polylactide as known in the art, for example.

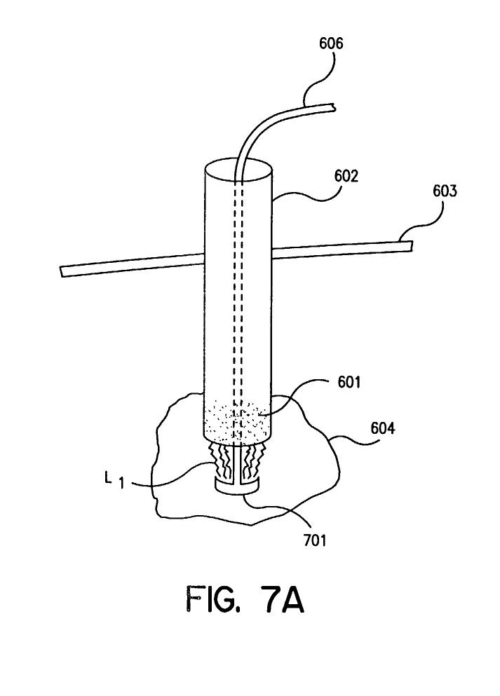

In another example of the invention as illustrated in FIGS. 6A and 6B, light-emitting nanoparticles 601 are incorporated into a polymeric sheath 602 for insertion beneath a patient's skin 603 in proximity to the treatment site 604. A removable needle 605 is optionally used to stiffen the sheath and to aid inserting the sheath into the site, as illustrated in FIG. 6A. The needle punctures the tissue to aid in positioning the sheath in the body, and the needle may also be used to inject the PDT drug if desired and optionally additional liquid-borne light-emitting nanoparticles into the treatment area. After the needle is withdrawn, leaving the sheath embedded in the target site, a light source such as an optical fiber 606 may be inserted into the sheath and used to activate the nanoparticles in the sheath and the optional liquid-borne nanoparticles, which in turn activate the PDT drugs at the treatment site as shown in FIG. 6B.

Optical fiber 606 may incorporate a cylindrical diffuser as described in U.S. Pat. No. 6,013,053. Upon illumination by a light source as described above, the nanoparticles emit light in a diffuse non-directional fashion and also emit light at desired wavelengths or wavebands capable of activating the PDT drug or drugs present at the site. After treatment, the matrix may either be removed or left in situ to biodegrade over time. The matrix is preferably composed of a biodegradable and bioresorbable material, such as polylactide. A preferred fiber, fabric or grid is formed of a biodegradable polymer as disclosed in the article by J. Middleton & A. Tipton, “Synthetic Biodegradable Polymers as Medical Devices,” Medical Plastics & Biomaterials, March/April 1998, 30-39. Preferably, the matrix is flexible to ease placement within or on the patient.

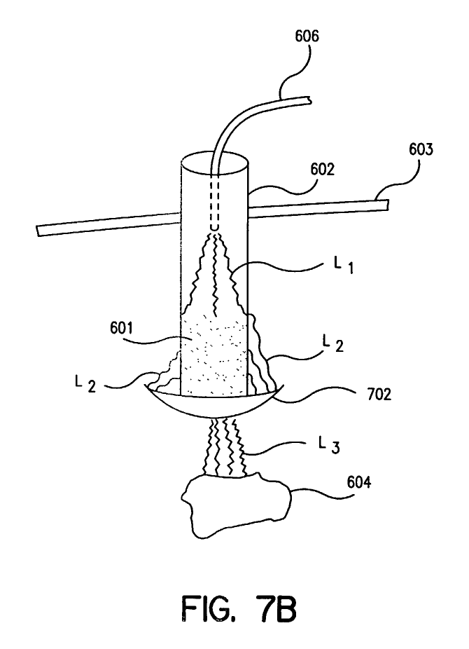

As illustrated in FIG. 7A, optical fiber 606 may incorporate a total-internal reflection (TIR) lens 701 rather than a diffuser. The TIR lens in this instance is receives light from the light source and is positioned in relation to the sheath 602 so that the lens focuses the light onto nanoparticles 601, which emit light to the treatment site 604.

FIG. 7B depicts how a TIR lens 702 may be incorporated into the sheath 602 rather than the optical fiber. Light L₁ from optical fiber 606 illuminates nanoparticles 601. The nanoparticles emit light L₂, some of which is captured by TIR lens 702 attached to sheath 602. Light L₂ is focused by TIR lens 702 to form light L₃, which illuminates target site 604.

In this instance, it is preferred that the light source generating light L₁ is not a laser but is instead e.g. a light-emitting diode array or other source of non-coherent or non-laser light. Light L₁ will spread to illuminate the quantum dots better. Thus, a non-coherent or non-laser light source is preferred in those instances where the surgeon prefers to have a light beam that diffuses after exiting the optical fiber.

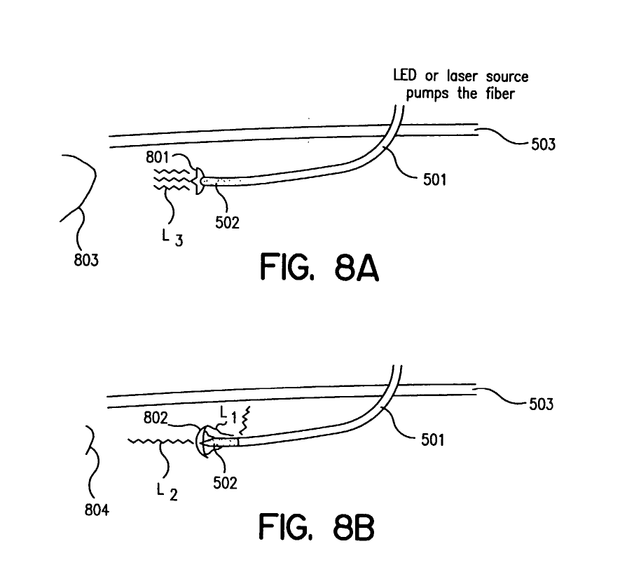

Other examples of the invention are shown in FIGS. 8A and 8B. An optical fiber 501 composed of a biocompatible matrix is shown. Quantum dots 502 coat the external shell of the fiber or are embedded in the matrix. The fiber is inserted beneath a patient's skin 503 in proximity to the PDT treatment site. An external light source, such as an LED, a laser, a laser diode, or other non-coherent light source such as a incandescent lamp or halogen light, pumps light into the fiber during treatment. In FIG. 8A, light L₃ from the quantum dot is spread over a wide area and is directed to diseased tissue 803. In FIG. 8B, light L₁ from the quantum dot is captured by total internal reflection (TIR) lens 802 and is concentrated as light beam L₂ onto or into the tissue 802 to be treated. As an alternative or additionally, the quantum dots may coat the TIR lens itself along portions of the TIR lens that form the optical pathway of the lens, so that as much of the light generated by the dots fluorescing is directed by the lens.

Suitable internal reflection lenses are disclosed in the following U.S. patents, which are incorporated herein in their entirety as if fully put forth below: U.S. Pat. No. 4,337,759, 5,404,869, 5,577,493, 5,613,769, 5,655,832, 5,676,453, 5,757,557, 5,806,955, 5,924,788, and 5,926,320. A total internal reflection lens is preferably configured so that it can be positioned within a patient to receive photodynamic therapy. For example, the TIR lens would be so configured if it met one or more of the following criteria: the lens is sterile; the lens is sufficiently small to be implanted within a patient's body; the lens has a size suitable for illuminating the target tissue (e.g. blood; tumor; or organ); the lens is configured to receive light from an optical fiber that itself is adapted for use within a patient. A total internal reflection lens may capture some of the light generated by light-emitting nanoparticles and emitted in a direction that would otherwise illuminate healthy areas if the TIR lens was not present. For example, light-emitting nanoparticles in a fiber optic sheath in sufficient proximity to the TIR lens to capture light that is emitted in a direction that would not illuminate the target site if the lens was not present. The TIR lens then emits this captured light at the target site. A total internal reflection lens can thus increase the amount of nanoparticle-emitted light that illuminates diseased tissue or cells.

Instead of focusing light, a TIR lens may be used to diffuse light over a larger area but in the direction of the diseased tissue. If e.g. an optical fiber or a light-emitting diode is placed at the focal point of the TIR lens rather than on the diffuse light-gathering side of the lens, the lens operates in reverse and diffuses the light to illuminate a broader area. Thus, light from the light source is spread over a larger area of the diseased tissue, or light emitted by nanoparticles embedded in a sheath is spread over a larger area of the diseased tissue.

In any of the embodiments above, light from the light source will also illuminate the target site. A site at which a PDT drug is activated by such light may thus be treated using a combination of PDT drugs, one or more which is activated by the light source directly and one or more which is activated by light emitted by the nanoparticles.

7. Administration of Light-Emitting Nanoparticles and PDT Drug

One embodiment of the present invention comprises administering a therapeutically effective amount of a PDT drug to a target area of a patient and also administering a sufficient quantity of light-emitting nanoparticles to the target area to provide an illuminance or fluence of light that activates the PDT drug.

PDT drugs are known in the art and are generally considered to be those drugs which absorb photons from a light source and transfer this energy to surrounding oxygen molecules. This in turn induces formation of singlet oxygen and other highly-reactive free radical species, leading to a number of biological effects, including damage to proteins, nucleic acids, lipids, and other cellular components. A PDT drug thus destroys cancer cells, cells responsible for atherosclerosis, psoriasis, or macular degeneration, or other bodies responsible for bacterial or viral infections. A PDT drug may instead be one which effects gene transcription.

The nanoparticles are irradiated with light of a first wavelength. Photons of this light have energy that is greater than or equal to the bandgap energy of the material forming the nanoparticles, so that the nanoparticles absorb this energy and subsequently re-emit energy in the form of light at a second wavelength. This light of a second wavelength is absorbed either by the PDT drug or by other nanoparticles than then reemit this light at a third wavelength. The third wavelength is absorbed by a PDT drug or is absorbed by other nanoparticies and re-emitted at a fourth wavelength that is absorbed by a PDT drug.

a) Administration of PDT Drug

A PDT drug or photosensitizing agent is a chemical compound which homes to one or more types of selected target cells and, when contacted by radiation, absorbs the light, which results in impairment or destruction of the target cells. Virtually any chemical compound that homes to a selected target and absorbs light may be used in this invention. Preferably, the chemical compound is nontoxic to the animal to which it is administered or is capable of being formulated in a nontoxic composition. Preferably, the chemical compound in its photodegraded form is also nontoxic. A comprehensive listing of photosensitive chemicals may be found in Kreimer-Birnbaum, Sem. Hematol. 26:157-73, 1989. Photosensitive compounds include, but are not limited to: indocyanine green (ICG); methylene blue; toluidine blue; aminolevulinic acid (ALA); phthalocyanines; porphyrins; texaphyrins; bacteriochlorins;, merocyanines, psoralens, benzoporphyrin derivatives (BPD) and porfimer sodium and pro-drugs such as α-aminolevulinic acid, which can produce drugs such as protoporphyrin. Also included are: chlorin compounds; purpurins; and any other agent that absorbs light in a range of 500 nm 1100 nm. More specifically, chlorin and purpurin compounds contemplated in the present invention, include: mono-, di-, or polyamide aminodicarboxylic acid derivatives of cyclic or non-cyclic tetrapyrroles (see: Bommer et al., U.S. Pat. Nos. 4,675,338 and 4,693,885); and alkyl ether derivatives of pyropheophorbide-a with N-substituted cyclic imides (purpurin-18 imides) (see: Pandy et al., WO 99/67249). Specifically, included are derivatives of mono-L-aspartyl chlorin e6 (NPe6); and any other agent that absorbs light in a range of 500 nm-1100 cm.

Other examples of PDT drugs include but are not limited to photosensitive compounds such as pheophorbides and pyropheophorbides. Porfimer sodium or omeprazole can be used to relieve symptoms of esophageal cancer. BPD verteporfin may be used for skin carcinoma, cutaneous lesions where cancer has metastasized to the skin, and chronic stable plaque psoriasis. BPD verteporfin is activated by red light having a wavelength of 690 nm and also by ultraviolet A light having a wavelength of 290-320 nm. Hematoporphyrin derivatives and especially dihematoporphyrin ether, which has a peak absorption at a wavelength of 405 nm and a low absorption peak at 630 nm, are also useful. PDT drugs include those that treat cancer of the bronchi, pharynx, oral cavity, bladder, eyes, and the uterus. Other examples of PDT drugs include Pharmacyclics' ANTRIN®, which may be used for photoangioplasty treatment of patients with peripheral vascular disease to remove arterial plaque; boronated porphyrin, being tested to treat brain cancer; Scotia QuantaNova's drug FOSCAN®, being tested to treat head and neck cancer; and QLT PhotoTherapeutics' PHOTOFRIN® (a precancerous condition of the esophagus).

A PDT drug often absorbs light at multiple wavelengths that are typically within a bandwidth of about 300 nm. Also, a number of drugs absorb light at a wavelength that is more difficult to deliver to the drug using current lasers or other light sources such as LEDs. Dihematoporphyrin ether absorbs light having a wavelength of 405 nm most effectively, yet it is not practical to deliver this shorter wavelength of light into the body because of the short penetration depth of light of this wavelength (on the order of a millimeter or less). Consequently, dihematoporphyrin ether is instead activated using light having a wavelength at which the drug absorbs more energy. Use of light-emitting nanoparticles that absorb light having a wavelength greater than 405 nm (e.g. 805 nm) and emit light at 405 nm is one option when practicing the invention. The 405 nm light emitted by the nanoparticles activates the dihematoporphyrin ether directly. Another option for this drug is to have both nanoparticles that emit light at 630 nm as well as nanoparticles that emit light at 405 nm together as part of the fluid that is administered to a patient and use a different wavelength or wavelengths of light from the light source to cause the nanoparticles to fluoresce.

A therapeutically effective amount of a PDT drug is administered to the patient. The drug is therefore present in the target site in an amount that produces a sufficient quantity of active species (such as singlet oxygen or other reactive species) to effect the particular therapy being administered. Only a fraction of the radicals or other reactive species generated by the drug damage critical cell sites to cause cell death or begin gene transcription or provide another beneficial effect. Consequently, the quantity of PDT drug is selected to assure that the beneficial effect of the drug is obtained. A typical dosage is between about 0.05 and 5 mg of PDT drug per kilogram of body weight.

Administration of PDT drugs to a patient may be systemic, by ingestion or injection; regional (e.g. to the gastrointestinal, hepatic, or renal systems, for example, or to anatomic regions such as the abdomen, lumbar spine, arm or leg regions), by intravenous injection or topical application; or by affinity targeting, by applying the compound to a specific treatment site on or in the patient's body via topical administration or injection into the target site, or by ingestion with a feeding tube. These and other possible methods of administration of PDT drugs are known to those skilled in the art and are included in the present invention.

b) Administration of Nanoparticles

A therapeutically effective amount of light emitting nanoparticles is also administered to the patient. Activating a PDT drug to effect a particular therapy (e.g. tumor or psoriasis treatment or gene transcription) requires a certain amount of light to reach the PDT drug. A quantity of light insufficient to effect therapy may cause some drug molecules to form reactive species such as single oxygen, but an insufficient number of the reactive species are generated to reduce tumor size or arrest tumor growth, for instance. A therapeutically-effective amount of nanoparticles is therefore a quantity that generates a sufficient amount of light to the PDT drug to produce the desired therapy, either alone or in combination with light supplied by the light source used to excite the nanoparticles to emit light. The amount of light and therefore the number of nanoparticles is a function of many factors, which include: the distance the nanoparticles are from the drug to be activated; the type and density of tissue, if any, in the optical path between the drug and the nanoparticles; the number of photons to which the drug is exposed, the wavelength of light used to activate the drug; the extinction coefficient of the PDT drug; the amount of PDT drug present; the length of time that the PDT drug is exposed to activating light; the oxygen concentration in the tissue neighboring the PDT drug (which affects the quantum yield of radicals); the fraction of radicals that actually contact the target site; and the optical penetration depth of the light that activates the PDT drug.

A sufficient quantity of nanoparticles emitting the activating wavelength is therefore provided to the target site sot that these nanoparticies are sufficiently close to the PDT drug to activate it. As noted previously, light of shorter wavelengths is readily absorbed by tissue. Consequently, a PDT drug that absorbs short wavelength light requires the light source to be very close to the PDT drug. Otherwise, tissue attenuates the light before it can activate the drug. Light emitting nanoparticles can be positioned sufficiently closely to PDT drug molecules to overcome the problem of tissue absorbing the light which activates the PDT drug.

A sufficient quantity of light-emitting nanoparticles close to the PDT drug can be provided in a number of ways. One way is to administer a sufficiently high number of nanoparticles to the patient systematically, regionally, or locally so that the desired concentration of nanoparticles is achieved in the vicinity of the PDT drug. Another way is to attach one or more delivery moieties to the nanoparticles that are specific to the target site area, so that the nanoparticles localize in the treatment area. The delivery moieties may bind specifically to a material produced by or found in the target area, or the delivery moieties may bind to the PDT drug. A third way to place nanoparticles within sufficient proximity to the PDT drug is to attach the nanoparticles to the drug either covalently or through an associating group, as described herein.

Thus, the light-emitting nanoparticles may be unmodified, may be solubilized, or may be attached to a delivery moiety as described above, thus targeting the nanoparticles to the treatment site. Nanoparticles attached to a delivery moiety have a wider time range over which they may be administered. Since often the delivery moiety is site-specific, nanoparticles having a delivery moiety are retained at the target site longer than nanoparticles not having a delivery moiety. Consequently, nanoparticles having a delivery moiety can be administered sooner than nanoparticles not having a delivery moiety. Further, the affinity that a delivery moiety has for the target site may allow such nanoparticles to be accumulated at a target site within a shorter period of time than nanoparticles without such groups require to accumulate at the site.

In one form of systemic PDT, the photosensitizing agent (also referred to as “PDT drug”) is injected into the blood-stream at time t₀. Cells within the body absorb the PDT drug. However, the PDT drug resides in disease cells (such as cancer cells) for a longer period of time than the drug resides in healthy or normal cells. Consequently, light administration is timed to occur at a time t₂ such that a sufficient amount of the PDT drug has dissipated to low levels in or near healthy cells. The PDT drug is also administered to the patient in an amount that results in a sufficient level of PDT drug in disease cells when the drug has dissipated sufficiently from healthy or normal cells such that PDT can commence without substantial harm to a significant number of healthy or normal cells.

A therapeutically effective quantity of nanoparticles may also be administered systematically to the patient by injecting them into the blood-stream. The nanoparticles are injected or otherwise systematically administered at a time t₁ to provide a sufficiently high concentration in sufficiently close proximity to the PDT drug to activate the drug at the appropriate time. Typically, the time t₁ occurs at or after time to, and usually the time t₁ occurs before t₂. Thus, the PDT drug and the nanoparticles may be administered simultaneously and allowed time to reach the treatment site before irradiating the site, or the nanoparticles may be administered after the drug is administered but before the time that the target site is irradiated.

The light-emitting nanoparticles may also be administered to the patient as light from the light source irradiates the target site, especially where the nanoparticles are injected locally into the patient or where the nanoparticles have delivery moieties that result in a sufficient number of the nanoparticles accumulating in the target area and activating the PDT drug to effect treatment. An area of the body will not receive photodynamic therapy so long as that area is not illuminated with light of a wavelength that causes the nanoparticles to fluoresce or does not contain a sufficiently high concentration of nanoparticles to cause significant amounts of PDT drug activation.

In systematic PDT drug administration, the time t₂ at which light is administered is typically one to three days after time t₀, the time at which the PDT drug is administered systematically, where the drug is used to treat cancers of the esophagus, bronchial tubes, cervix, pancreas, or colon for instance. The time t₁ at which nanoparticles are administered in these instances is typically between 1 hour and 72 hours after the time at which the PDT drug is administered.

If the PDT drug is one used to treat wet macular degeneration, the time t₂ at which time is administered is typically several minutes after the photosensitive drug has been administered intravenously. Nanoparticles may be administered at any time t₁ before time t₂ so long as there is sufficient time to build a sufficient concentration of the nanoparticles in close proximity to the photoactive drug at the site to be treated. The rapidly-growing, new blood vessels which grow beneath the retina and bleed are treated, whereas the normal vessels are essentially undisturbed.

Multiple PDT drugs may be administered and/or activated simultaneously or sequentially. Consequently, two or more PDT drugs may be administered separately but allowed sufficient time to accumulate at a treatment site, then the drugs are activated simultaneously when the light source illuminates the target area and when sufficient light-emitting nanoparticles are present. Further, the drugs may be activated separately by supplying a mixture of nanoparticles (one type of nanoparticle that emits wavelength λ₁ and activates drug number 1, a second type that emits wavelength λ₂ and activates drug number 2, and so forth) and by irradiating the area with activating light from a light source that supplies a first wavelength of actinic light sufficient to cause the nanoparticles to emit λ₁, then irradiating the area with activating light that supplies a second wavelength sufficient to cause the nanoparticles to emit λ₂ and so forth.

c) Light Activation

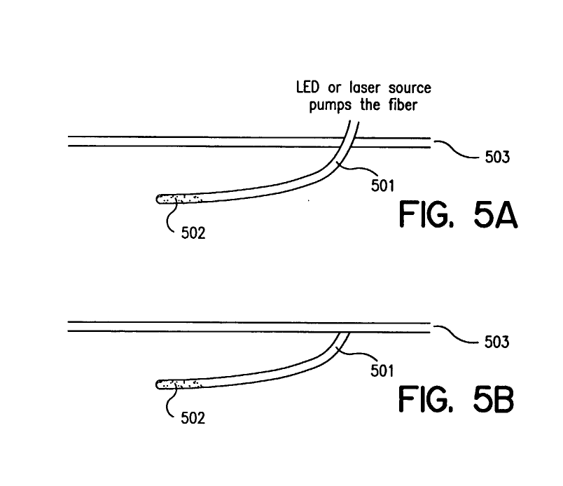

As illustrated in FIG. 4, as or after a sufficient number of nanoparticles accumulate at the treatment site, a light source 401 is introduced into a treatment site 402 beneath the skin 403, or a light source 404 is applied to the patient's skin externally to generate light waves 405 that irradiate light-emitting nanoparticles 201 attached to site-specific antigens 202. The light source may be any suitable source which provides light that is absorbed by the nanoparticles structure and which optionally penetrates tissue. Light sources that may be used in accordance with the present intention include but are not limited to lasers and laser diodes, light emitting diodes (LEDs) formed of semiconductor or organic materials, electroluminescent light sources, incandescent light sources, cold cathode fluorescent light sources, and other light sources emitting light of a wavelength capable of causing the light-emitting nanoparticles to fluoresce. Tissue-penetrating light sources typically emit one or more wavelengths within the 350-1100 nm waveband, and preferably emit one or more wavelengths within the 600-100 nm waveband.

Light may be delivered subcutaneously by coupling the light to a fiber optic and directing it through e.g. a bronchoscope into the lungs for the treatment of lung cancer, through an endoscope into the esophagus for the treatment of esophageal cancer, or through a catheter into or near the tumor at another location within the body. One preferred light source is a portable light source that a patient may wear continuously through the treatment period, as disclosed in U.S. Ser. No. 09/232,129, “Patient Portable Device For Photodynamic Therapy,” James Chen et al., filed Jan. 15, 1999 (also disclosed in PCT/US00/00805, “Patient Portable Device For Photodynamic Therapy,” James Chen et al., filed Jan 14, 2000).

A preferred internal light source is a laser coupled to an optical fiber. The fiber may have a diffusing tip attached, such as a cylindrically-shaped or spherically-shaped diffuser. A preferred light source is an external LED array focused with a totally internally reflecting lens, such as that disclosed in WO 98/33251 and WO 98/20936.

Wavelengths and wavebands longer than 600 nm are preferred for an external light source, because they are capable of penetrating tissue fairly deeply. Light at a wavelength of 630 nm effectively penetrates approximately 3 cm into tissue before the optical energy has been dissipated. An external light source is useful for treating psoriasis, skin carcinomas, and other disorders in which the target site is located within approximately 3 cm of the skin surface. Other wavelengths include those at which two-photon activation occurs (e.g. between 750 and 1240 nm, and especially about 800 nm).

A light source typically produces a range of wavelengths (“waveband”). A laser produces a narrow waveband of light, whereas an LED array produces a broad waveband or multiple wavebands of light. The waveband of the light source is selected to stimulate emission from the light-emitting nanoparticies selected for the particular therapy to be administered.

It is often difficult to provide light of wavelengths suitable to activate one or more drugs at multiple wavelengths. As noted above, a laser produces wavelengths within a narrow waveband. Consequently, multiple lasers are needed to activate a drug or multiple drugs at multiple wavelengths. Further, LEDs are not yet commercially available that emit in different waveband ranges. LEDs that emit in the blue light region are currently being commercialized, but LEDs having shorter wavelengths in the blue-violet, violet, and ultraviolet wavelength range are not readily available. The use of light-emitting nanoparticles enables a surgeon to irradiate a treatment site with narrow-band light or light having multiple wavebands and convert that light into light of other wavelengths and wavebands. It is thus possible to convert light of a narrow waveband into broadband emission or into light emission of multiple desired wavebands specific to the drug or drugs selected for use in PDT by selecting the light-emitting nanoparticles accordingly.

The activating light source emits light that the nanoparticles absorb. Consequently, if the nanoparticles all absorb light at about the same wavelength, a narrow-band light source such as a laser can be used. Or, if the nanoparticles absorb light at different wavelengths, a broader-band light source may be used such an LED array. Light-emitting nanoparticles may themselves be a light source for other light-emitting nanoparticles as described previously in order to allow a light source to be used that produces light of a wavelength that is absorbed by some of the nanoparticles but not all of them. In this case, the nanoparticles acting as a light source for other nanoparticles absorb the actinic radiation from the light source, then emit radiation at a second wavelength that at least some of the other nanoparticles absorb, causing them to fluoresce. In another preferred embodiment of the invention, a narrow-band light source such as a laser is used to activate a mixture of nanoparticles whose emission provides a broad band of light.

In one embodiment of the invention, the wavelength range emitted from the external or internal light source is selected such that it does not activate the PDT drug directly. For example, the light source can be selected to produce light having a wavelength within the range of about 600 or 800 nm to about 1100 nm, which does not activate some presently-available PDT drugs effectively. The light source activates the nanoparticles, and the nanoparticles in turn re-emit the light as fluorescence at a wavelength or waveband that closely matches the absorption band(s) of the PDT drug (e.g. at a wavelength of 405 nm and 630 nm for dihematoporphyrin ether). The drug is thus activated by way of the photoluminescence from the nanoparticles. Excitation of the drug in turn leads to formation of singlet oxygen and other reactive species and result in cell death. Combinations of different types of PDT drugs and/or different types of nanoparticles may be used simultaneously to achieve desired effects for a particular condition or treatment.

8. Other Preferred Therapies

One preferred therapy involves administering light-emitting nanoparticles and PDT Drug that are bound to one another either directly or through a backbone polymer and illuminating a target site in a patient to cause the nanoparticles to fluoresce. A sufficient number of light-emitting nanoparticles are bound to the PDT drug so that they are in the vicinity of the drug during use and illuminate the drug, thus producing singlet oxygen. The nanoparticles are illuminated using either a light source external to the patient or one internal to the patient.

Another preferred therapy involves administering a PDT drug and light-emitting nanoparticles that are attached to one or more types of delivery moieties, and illuminating the target site with light that causes the nanoparticles to fluoresce after the nanoparticles are given sufficient time to associate or bind with the target site. The target site has proteins or other molecules, cells, tissue, or other materials for which the delivery moieties have an affinity, as described previously.

In one therapy, the nanoparticles are selected to provide a desired intensity of light at a given wavelength. A mixture of nanoparticles as described above (e.g. mixtures of different sizes and/or compositions; mixtures of quantum dots, nanocrystals, and/or quantum rods) is formed to provide the desired light intensities at various wavelengths, and the mixture is administered to the patient to activate either a single PDT drug that absorbs light at multiple wavelengths or a mixture of PDT drugs, any of which absorbs light at a single wavelength or multiple wavelengths.

In another preferred embodiment of the invention, a mixture of nanoparticles is administered that provides multiple wavebands of light. The wavebands are selected to provide light of a wavelength that is absorbed by a PDT drug that has also been administered to the patient. The mixture is also optionally formed to provide different light intensities for the different wavebands present, so that the intensity varies as a function of the absorption peak for the PDT drug or drugs used in the therapy. Consequently, the particle size and type (e.g. quantum dots, nanocrystals, and/or quantum rods) of the nanoparticles are selected to provide the desired wavebands and waveband intensities.

Any of the components, compositions, drugs, and tools discussed herein as being placed within a patient are preferably sterile or otherwise suitable for placement within the patient. Thus, any optic fiber, TIR lens, nanoparticles, and PDT drug to be placed within a patient are preferably sterile. Further, there may be packaged combinations of these materials. For example, an optic fiber may be packaged with nanoparticles, where the nanoparticles are separate from the optic fiber or are unitary with (e.g. embedded in, fixed upon, or otherwise part of) the optic fiber. Nanoparticles may be packed with a PDT drug or combination of drugs that is activated at the wavelength or waveband of light emitted by the nanoparticles. Further, the PDT drug may be a combination of drugs that activate at different wavelengths (such as at wavelengths emitted by the nanoparticles and optionally at the wavelength emitted by the light source). Any of the components or kits of components may also contain a set of instructions for use of the components as are utilized in the medical field to instruct a surgeon on use of the component or components. Thus, for example, a PDT drug may have instructions on dosage and administration of the drug. An optic fiber may have instructions for insertion and/or use within a patient. Nanoparticles may be accompanied by instructions to use only with a particular PDT drug.

The invention thus provides new or improved tools, compositions, and methods associated with treatment of disease, and the invention is intended to cover all such new or improved tools, and methods disclosed herein.

What is claimed is:

1. A composition for administration to a patient that is useful in photodynamic therapy in the patient, said composition comprising a carrier and a plurality of light-emitting nanoparticles that emit light having a first wavelength which activates a photodynamic therapy drug which absorbs light of said first wavelength, said light-emitting nanoparticles being present in said carrier in a quantity that is effective to activate said photodynamic therapy drug upon illumination with light from a light source emitting a second wavelength of light.

2. A composition according to claim 1, wherein said light emitting nanoparticles fluoresce upon absorption of two photons of activating light.

3. A composition according to claim 1 further comprising a therapeutically effective amount of said photodynamic therapy drug.

4. A composition according to claim 1 that emits multiple wavelengths of light within a waveband, wherein at least a portion of said light emitting nanoparticles are formed of a first light-emitting material and have different particle sizes.

5. A composition according to claim 1 that emits multiple wavebands of light, wherein a first portion of said light emitting nanoparticles are formed of a first light-emitting material and have different particle sizes that generate first wavelengths of a first waveband, and a second portion of said light emitting nanoparticles are formed of a second light-emitting material and have different particle sizes that generate second wavelengths of a second waveband.

6. A composition according to claim 1, wherein at least a portion of said plurality of light-emitting nanoparticles individually further comprise a target-specific component.

7. A conjugate according to claim 6, wherein the target-specific component is a first member of an affinity binding set, wherein said first member is capable of selectively binding to a second member of the affinity binding set.

8. A conjugate according to claim 7, wherein the affinity binding set is selected from the group consisting of: biotin-avidin; biotin-streptavidin; antibody-antigen; antibody fragment-antigen; chemokine-chemokine receptor; and growth factor-growth factor receptor.

9. A mixture of light emitting nanoparticles adapted for use in photodynamic therapy, wherein said mixture comprises a first plurality of light-emitting nanoparticles that absorb light at a first wavelength and emit light at a second wavelength; a second plurality of light-emitting nanoparticles that absorb light at a third wavelength and emit light at fourth wavelength, said fourth wavelength differing from said second wavelength; and a carrier that is suitable to administer to a human patient; wherein said first plurality of light-emitting nanoparticles is present in said carrier in a sufficiently large number to activate a first PDT drug molecule upon administration to said patient; and wherein said second plurality of light-emitting nanoparticles is present in said carrier in a sufficiently large number to activate a second PDT drug molecule upon administration to said patient.

10. A conjugate for administration to a patient that is useful in photodynamic therapy in the patient, said conjugate comprising a light-emitting nanoparticle linked to a photodynamic therapy drug.

11. A conjugate according to claim 10, wherein said nanoparticle fluoresces upon absorption of two photons of activating light.

12. A conjugate providing sufficient illuminance to activate a photosensitive drug, said conjugate comprising a plurality of light-emitting nanoparticles linked to a polymeric backbone.

13. A conjugate according to claim 12, wherein said conjugate further comprises a target-specific component.

14. A method for photodynamic therapy, comprising:
administering a therapeutically effective amount of a photosensitive drug to a treatment area, wherein said photosensitive drug absorbs light of a first wavelength;
administering a therapeutically effective quantity of light-emitting nanoparticles to said treatment area, wherein said nanoparticles absorb light of a second wavelength and emit light of said first wavelength and wherein said therapeutically effective quantity of the light-emitting nanoparticles provides sufficient light of the first wavelength upon irradiation with light of the second wavelength to cause the photosensitive drug to effect said therapy; and
illuminating the treatment area with light from a light source that emits light of said second wavelength, thereby causing the light-emitting nanoparticles to emit light of the first wavelength.

15. A method according to claim 14, wherein the photosensitive compound is selected from the group consisting of indocyanine green; methylene blue; toluidine blue; aminolevulinic acid; phthalocyanines; porphyrins; texaphyrins; bacteriochlorins; merocyanines; psoralens; benzoporphyrin derivatives; porfimer sodium and its pro-drugs; π-aminolevulinic acid; protoporphyrin; chlorin compounds; purpurins; mono-, di-, or polyamide aminodicarboxylic acid derivatives of cyclic or non-cyclic tetrapyrroles; alkyl ether derivatives of pyropheophorbide-a with N-substituted cyclic imides; derivatives of mono-L-aspartyl chlorin e6 (NPe6); pheophorbides and pyropheophorbides; porfimer sodium; omeprazole; benzoporphyrin verteporfin; hepatoporphyrin derivatives; and dihematoporphyrin ether.

16. A method according to claim 14, wherein the nanoparticles fluoresce upon absorbing two photons of said light of said second wavelength.

17. A method according to claim 14, wherein the nanoparticles are linked to a polymer backbone.

18. A method according to claim 14, wherein the light source is selected from the group consisting of a laser diode and a light emitting diode.

19. A method according to claim 14, wherein the light source comprises a laser coupled to an optical fiber.

20. A method according to claim 14, wherein the light source comprises a light emitting diode array focused with a lens system.

21. A method according to claim 20, wherein the lens system comprises a totally internally reflecting lens.

22. A method according to claim 14 wherein the act of illuminating the treatment area with light from said light source that emits said light of said second wavelength, thereby causing the light-emitting nanoparticles to emit said light of the first wavelength, comprises:
illuminating a total internal reflection lens with said light from the light source and illuminating the nanoparticles with light transmitted by the total internal reflection lens.

23. A method according to claim 22, wherein the total internal reflection lens receives said light from the light source in an area of said lens having first cross-sectional area and transmits said light from the lens over an area greater than the first cross-sectional area.

24. A method according to claim 22, wherein the total internal reflection lens receives said light from the light source in an area of said lens having a first cross-sectional area and transmits said light from the lens over an area less than the first cross-sectional area.

25. A method according to claim 14 wherein the act of illuminating the treatment area with light from said light source that emits said light of said second wavelength, thereby causing the light-emitting nanoparticles to emit said light of the first wavelength, comprises:
illuminating a total internal reflection lens with the light of the first wavelength generated by the nanoparticles, and illuminating the treatment area with light transmitted by the total internal reflection lens.

26. A method according to claim 25, wherein the total internal reflection lens receives said light from the light source in an area of said lens having a first cross-sectional area and transmits said light from the lens over an area greater than the first cross-sectional area.

27. A method according to claim 25, wherein the total internal reflection lens receives said light from the light source in an area of said lens having a first cross-sectional area and transmits said light from the lens over an area less than the first cross-sectional area.

28. A method for photodynamic therapy, comprising administering a therapeutically effective amount of a conjugate to a patient, wherein said conjugate comprises a light-emitting nanoparticle linked to a photosensitive compound.

29. A method according to claim 28, wherein the nanoparticle fluoresces upon absorbing two photons of activating light.

30. A method for photodynamic therapy, comprising:
administering a therapeutically effective amount of a photosensitive compound to a patient; and
administering a biocompatible matrix composed of a solid material to a treatment area of said patient, wherein said matrix includes a therapeutically effective quantity of nanoparticles.

31. A method according to claim 30, wherein at least a portion of said quantity of nanoparticles fluoresces upon absorbing two photons of light of a first wavelength.

32. An illumination system for activating a photodynamic therapy drug, said illumination system comprising
a) a light source;
b) an optical fiber positioned to receive light generated by the light source and transmit said light toward an end of the optical fiber; and
c) a total internal reflection lens positioned so that said lens receives light from the optical fiber and transmits light from said lens.

33. An illumination system according to claim 32 wherein said illumination system further comprises light-emitting nanoparticles that emit light having a first wavelength when illuminated by light of a second wavelength, said nanoparticles being positioned in an optical path of the illumination system such that said illumination system emits said light having the first wavelength.

34. An illumination system according to claim 33, wherein said nanoparticles are positioned to be illuminated by the light from the optical fiber.

35. An illumination system according to claim 33 wherein said nanoparticles are unitary with said optical fiber.

36. An illumination system according to claim 33 wherein said total internal reflection lens is positioned to receive said light from said nanoparticles across a first cross-sectional area of said lens and said total internal reflection lens transmits light from a second cross-sectional area of said lens, said first cross-sectional area being greater than said second cross-sectional area.

37. An illumination system according to claims 35 wherein said total internal reflection lens is positioned to receive said light from said nanoparticles across a first cross-sectional area of said lens and said total internal reflection lens transmits light from a second cross-sectional area of said lens, said first cross-sectional area being greater than said second cross-sectional area.

38. An illumination system according to claim 33 wherein said total internal reflection lens is positioned to receive said light from said nanoparticles across a first cross-sectional area of said lens and said total internal reflection lens transmits light from a second cross-sectional area of said lens, said first cross-sectional area being less than said second cross-sectional area.

39. An illumination system according to claim 35 wherein said total internal reflection lens is positioned to receive said light from said nanoparticles across a first cross-sectional area of said lens and said total internal reflection lens transmits light from a second cross-sectional area of said lens, said first cross-sectional area being less than said second cross-sectional area.

40. An illumination system according to claim 33, wherein said nanoparticles are positioned to be illuminated by the light transmitted from the total internal reflection lens.

41. An illumination system according to claim 40 wherein said total internal reflection lens is positioned to receive said light from said nanoparticles across a first cross-sectional area of said lens and said total internal reflection lens transmits light from a second cross-sectional area of said lens, said first cross-sectional area being greater than said second cross-sectional area.

42. An illumination system according to claim 40 wherein said total internal reflection lens is positioned to receive said light from said nanoparticles across a first cross-sectional area of said lens and said total internal reflection lens transmits light from a second cross-sectional area of said lens, said first cross-sectional area being less than said second cross-sectional area.

43. An illumination system for activating a photodynamic therapy drug, said illumination system comprising:
a) a light source adapted to transmit light of a second wavelength to a treatment site of a patient; and
b) a quantity of nanoparticles that emit light having a first wavelength upon illumination by said light source.

44. An illumination system according to claim 43 wherein said illumination system further comprises a total internal reflection lens which receives light of said second wavelength to transmit said light to said nanoparticles.

45. An illumination system according to claim 43 wherein said illumination system further comprises a total internal reflection lens which receives a light of said first wavelength to transmit said light to said treatment site.

46. An illumination system according to claim 43 wherein said light source comprises an optical fiber that transmits said light having the first wavelength to the treatment site.

47. A kit for photodynamic therapy in a patient, wherein the kit comprises a packaged combination of light-emitting nanoparticles that emit light of a first wavelength when illuminated by light of a second wavelength and an optical fiber which transmits light of said second wavelength.

48. A kit for photodynamic therapy in a patient, wherein the kit comprises a packaged combination of light-emitting nanoparticles that emit light of a first wavelength when illuminated by light of a second wavelength and a first photodynamic therapy drug that is activated by the light of the first wavelength.

49. A kit according to claim 48 wherein the kit further comprises a second photodynamic therapy drug that is activated by the light of the second wavelength.

50. A kit according to claim 47 and further comprising a set of instructions on use of the kit.

51. A composition according to claim 1, wherein said light-emitting nanoparticles comprise quantum rods.

52. A composition according to claim 1, wherein said light-emitting nanoparticles comprise TiO₂.

53. A composition according to claim 1, wherein said light-emitting nanoparticles produce light having a sufficient illuminance at a wavelength of less than 600 nm to activate said photodynamic therapy drug.

54. A composition according to claim 1, wherein said lightemitting nanoparticles produce light having a sufficient illuminance at a wavelength of less than 500 nm to activate said photodynamic therapy drug.

55. A composition according to claim 1, wherein said light emitting nanoparticles absorb light having a wavelength greater than 600 nm.

56. A composition according to claim 1, wherein said light emitting nanoparticles absorb light having a wavelength greater than 700 nm.

57. A composition according to claim 1, wherein said light emitting nanoparticles absorb light having a wavelength greater than 800 nm.

58. A composition according to claim 1, wherein said light emitting nanoparticles fluoresce upon absorption of two photons of activating light.

59. A composition according to claim 1 further comprising a therapeutically effective amount of said photodynamic therapy drug.

60. A composition according to claim 1, wherein said carrier comprises a biocompatible liquid.

61. A composition according to claim 1, wherein said carrier comprises a film.

62. A composition according to claim 1, wherein said carrier comprises a biodegradable polymer.

63. A composition according to claim 1 that emits multiple wavelengths of light within a waveband, wherein at least a portion of said light emitting nanoparticles are formed of a first light-emitting material and have different particle sizes.

64. A composition according to claim 1 that emits multiple wavebands of light, wherein a first portion of said light emitting nanoparticles are formed of a first light-emitting material and have different particle sizes that generate first wavelengths of a first waveband, and a second portion of said light emitting nanoparticles are formed of a second light-emitting material and have different particle sizes that generate second wavelengths of a second waveband.

65. A composition according to claim 1, wherein at least a portion of said plurality of light-emitting nanoparticles individually further comprise a target-specific component.

66. A conjugate according to claim 65, wherein the target-specific component is a first member of an affinity binding set, wherein said first member is capable of selectively binding to a second member of the affinity binding set.

67. A conjugate according to claim 66, wherein the affinity binding set is selected from the group consisting of: biotin-avidin; biotin-streptavidin; antibody-antigen; antibody fragment-antigen; chemokine-chemokine receptor; and growth factor-growth factor receptor.

68. A mixture according to claim 69, wherein said first wavelength and said third wavelength are equal.

69. A mixture according to claim 9, wherein the difference between said first wavelength and said third wavelength is no more than 300 nm.

70. A mixture according to claim 9, wherein said carrier comprises a biocompatible liquid.

71. A mixture according to claim 9, wherein said carrier comprises a solid material.

72. A mixture according to claim 71, wherein the solid material comprises a biodegradable material.

73. A mixture according to claim 9, wherein said mixture further comprises said first PDT drug molecule and said second PDT drug molecule.

74. A mixture according to claim 73, wherein said first PDT drug molecule and said second PDT drug molecule are identical.

75. A mixture according to claim 9, wherein each member of at least a portion of said first plurality of light-emitting nanoparticles individually has a delivery moiety.

76. A mixture according to claim 9, wherein at least a portion of said first plurality of light-emitting nanoparticles are attached to a polymer.

77. A conjugate according to claim 10, wherein said nanoparticle is linked to said photodynamic therapy drug through a target-specific component.

78. A conjugate according to claim 77, wherein the target-specific component is a first member of an affinity binding set, wherein said first member is capable of selectively binding to a second member of the affinity binding set.

79. A conjugate according to claim 78, wherein the affinity binding set is selected from the group consisting of: biotin-avidin; biotin-streptavidin; antibody-antigen; antibody fragment-antigen; chemokinechemokine receptor; and growth factor-growth factor receptor.

80. A conjugate according to claim 10, wherein the nanoparticle and the photosensitive drug are individually linked to a polymeric backbone.

81. A conjugate according to claim 80, further comprising at least one additional nanoparticle to provide a plurality of nanoparticles in said conjugate.

82. A conjugate according to claim 81, wherein said plurality of nanoparticles provides light of sufficient illuminance to activate said photosensitive drug.

83. A method according to claim 14, wherein the nanoparticles comprise quantum rods.

84. A method according to claim 14, wherein the nanoparticles are a portion of a film that is administered to the treatment area.

85. A method according to claim 14, wherein the nanoparticles are incorporated into liposomes.

86. A method according to claim 14, wherein the nanoparticles are incorporated into nanocapsules.

87. A method according to claim 86, wherein the nanocapsules have a target-specific component.

88. A method according to claim 87, wherein the target-specific component is located on outer surfaces of the nanocapsules and comprises a first member of an affinity binding set, wherein said first member is capable of selectively binding to a second member of the affinity binding set.

89. A method according to claim 88, wherein the affinity binding set is selected from the group consisting of: biotin-avidin; biotin-streptavidin; antibody-antigen; and antibody fragment-antigen.

90. A method according to claim 14, wherein the nanoparticles are composed of a biocompatible material.

91. A method according to claim 90, wherein the biocompatible material comprises TiO₂.

92. A method according to claim 14, wherein the nanoparticles are linked to a target-specific component.

93. A method according to claim 92, wherein the target-specific component is a first member of an affinity binding set, wherein said first member is capable of selectively binding to a second member of the affinity binding set.

94. A method according to claim 93, wherein the affinity binding set is selected from the group consisting of: biotin-avidin; biotin-streptavidin; antibody-antigen; and antibody fragment-antigen.

95. A method according to claim 14, wherein the second wavelength is greater than about 600 nm.

96. A method according to claim 95, wherein the second wavelength is greater than about 700 nm.

97. A method according to claim 95, wherein the second wavelength is greater than about 800 nm.

98. A method according to claim 14, wherein the act of illuminating the treatment area consists essentially of introducing light of said second wavelength subcutaneously at the treatment area.

99. A method according to claim 14, wherein the act of illuminating the treatment area comprises irradiating an external portion of the patient's body at the treatment area with light of the second wavelength.

100. A method according to claim 98, wherein the light source is selected from the group consisting of a laser diode and a light emitting diode.

101. A method according to claim 98, wherein the light source comprises a laser coupled to an optical fiber.

102. A method according to claim 101, wherein the light source further comprises a light-diffusing tip positioned at an end of the optical fiber that is inserted into the patient.

103. A method according to claim 102, wherein the diffusing tip is cylindrical.

104. A method according to claim 15, wherein the light source comprises a light emitting diode array focused with a lens system.

105. A method according to claim 104, wherein the lens system comprises a totally internally reflecting lens.

106. A method according to claim 28, wherein the conjugate further comprises a linked target-specific component.

107. A method according to claim 106, wherein the target-specific component is a first member of an affinity binding set, wherein said first member is capable of selectively binding to a second member of the affinity binding set.

108. A method according to claim 107, wherein the affinity binding set is selected from the group consisting of: biotin-avidin; biotin-streptavidin; antibody-antigen; and antibody fragment-antigen.

109. A method according to claim 30, further comprising illuminating the treatment area with light having a first wavelength which causes the nanoparticles to emit light having a second wavelength that activates the photosensitive compound.

110. A method according to claim 30, wherein the biocompatible matrix is biodegradable.

111. A method according to claim 30, wherein the biocompatible matrix is composed of polylactide.

112. A method according to claim 30, wherein the biocompatible matrix is administered by inserting said matrix into the patient's body.

113. A method according to claim 30, wherein both the photosensitive compound and the nanoparticles form part of the biocompatible matrix.

114. A method for photodynamic therapy according to claim 30, wherein the act of administering the biocompatible matrix comprises inserting a polymeric sheath into said patient's body, said polymeric sheath having proximal and distal ends and having nanoparticles incorporated on or in the polymeric material of which the sheath is composed, such that the distal end is located in proximity to the treatment area after insertion.

115. A method according to claim 114, further comprising illuminating the treatment area with a light source that emits light having a first wavelength which causes the nanoparticles to emit light having a second wavelength that activates the photosensitive compound.

116. A method according to claim 115, wherein the polymeric sheath contains a removable needle.

117. A method according to claim 116, further comprising delivering the photosensitive compound to the treatment area by injecting it into the patient's body and in proximity to the treatment area through the removable needle.

118. A method according to claim 116, wherein the method further comprises removing the needle after insertion of the polymeric sheath into the patient, and wherein the light source comprises a fiber optic light source that is inserted into the sheath from the proximal end of the sheath to the distal end.

119. A method according to claim 14 wherein the act of illuminating the treatment area with light from said light source that emits said light of said second wavelength, thereby causing the light-emitting nanoparticles to emit said light of the first wavelength, comprises: illuminating a total internal reflection lens with said light from the light source and illuminating the nanoparticles with light transmitted by the total internal reflection lens.

120. A method according to claim 119, wherein the total internal reflection lens receives said light from the light source in an area of said lens having a first cross-sectional area and transmits said light from the lens over an area greater than the first cross-sectional area.

121. A method according to claim 119, wherein the total internal reflection lens receives said light from the light source in an area of said lens having a first cross-sectional area and transmits said light from the lens over an area less than the first cross-sectional area.

122. A method according to claim 14 wherein the act of illuminating the treatment area with light from said light source that emits said light of said second wavelength, thereby causing the light-emitting nanoparticles to emit said light of the first wavelength, comprises: illuminating a total internal reflection lens with the light of the first wavelength generated by the nanoparticles, and illuminating the treatment area with light transmitted by the total internal reflection lens.

123. A method according to claim 122, wherein the total internal reflection lens receives said light from the light source in an area of said lens having a first cross-sectional area and transmits said light from the lens over an area greater than the first cross-sectional area.

124. A method according to claim 122, wherein the total internal reflection lens receives said light from the light source in an area of said lens having a first cross-sectional area and transmits said light from the lens over an area greater than the first cross-sectional area.

125. A method according to claim 28 wherein the act of illuminating the treatment area with light from said light source that emits said light of said second wavelength, thereby causing the light-emitting nanoparticles to emit said light of the first wavelength, comprises: illuminating a total internal reflection lens with said light from the light source and illuminating the nanoparticles with light transmitted by the total internal reflection lens.

126. A method according to claim 125, wherein the total internal reflection lens receives said light from the light source in an area of said lens having a first cross-sectional area and transmits said light from the lens over an area greater than the first cross-sectional area.

127. A method according to claim 125, wherein the total internal reflection lens receives said light from the light source in an area of said lens having a first cross-sectional area and transmits said light from the lens over an area less than the first cross-sectional area.

128. A method according to claim 28 wherein the act of illuminating the treatment area with light from said light source that emits said light of said second wavelength, thereby causing the light-emitting nanoparticles to emit said light of the first wavelength, comprises: illuminating a total internal reflection lens with the light of the first wavelength generated by the nanoparticles, and illuminating the treatment area with light transmitted by the total internal reflection lens.

129. A method according to claim 128, wherein the total internal reflection lens receives said light from the light source in an area of said lens having a first cross-sectional area and transmits said light from the lens over an area greater than the first cross-sectional area.

130. A method according to claim 128, wherein the total internal reflection lens receives said light from the light source in an area of said lens having a first cross-sectional area and transmits said light from the lens over an area greater than the first cross-sectional area.

131. A method according to claim 30 wherein the act of illuminating the treatment area with light from said light source that emits said light of said second wavelength, thereby causing the light-emitting nanoparticles to emit said light of the first wavelength, comprises: illuminating a total internal reflection lens with said light from the light source and illuminating the nanoparticles with light transmitted by the total internal reflection lens.

132. A method according to claim 131, wherein the total internal reflection lens receives said light from the light source in an area of said lens having a first cross-sectional area and transmits said light from the lens over an area greater than the first cross-sectional area.

133. A method according to claim 131, wherein the total internal reflection lens receives said light from the light source in an area of said lens having a first cross-sectional area and transmits said light from the lens over an area less than the first cross-sectional area.

134. A method according to claim 30 wherein the act of illuminating the treatment area with light from said light source that emits said light of said second wavelength, thereby causing the light-emitting nanoparticles to emit said light of the first wavelength, comprises: illuminating a total internal reflection lens with the light of the first wavelength generated by the nanoparticles, and illuminating the treatment area with light transmitted by the total internal reflection lens.

135. A method according to claim 134, wherein the total internal reflection lens receives said light from the light source in an area of said lens having a first cross-sectional area and transmits said light from the lens over an area greater than the first cross-sectional area.

136. A method according to claim 134, wherein the total internal reflection lens receives said light from the light source in an area of said lens having a first cross-sectional area and transmits said light from the lens over an area greater than the first cross-sectional area.
